(19) United States
(12) Patent Application Publication
(10) Pub. No.: US 2008/0190421 A1
(43) Pub. Date: Aug. 14, 2008

(54) Venturi apparatus with incorporated flow metering device

(76) Inventor: Darryl Zitting, Salt Lake City, UT (US)

Correspondence Address: FACTOR & LAKE, LTD, 1327 W. WASHINGTON BLVD., SUITE 5G/H, CHICAGO, IL, 60607, US

(21) Appl. No.: 11/705,293

(22) Filed: Feb. 12, 2007

(51) Int. Cl. A61M 16/00

(52) U.S. Cl. 128/200.24

(57) ABSTRACT

A gas delivery apparatus for supplying an oxygen/air mix to a patient is discussed. Besides the conventional ability to adjust oxygen/air ratio independently via changes in the primary and/or secondary oxygen flow tube's flow rate adjustments, a flow meter is provided in the primary oxygen flow tube to give an accurate reading of the total flow of the oxygen/air mix exiting the venturi chamber of the apparatus.

### FIELD OF THE INVENTION

This invention relates to a gas mixing device capable of delivering oxygen or oxygen/air mix to a patient with a flow meter incorporated therein to enable a user to determine the total flow of oxygen/air mix being received.

### BACKGROUND OF THE INVENTION

Gas delivery devices for diluting the concentration of oxygen are well known. These devices generally comprise nozzles for securing a primary oxygen supply tube and a secondary oxygen supply tube, and a venturi portion into which oxygen is directed from the nozzles. The venturi portion generally has an air inlet in communication with an ambient air, directly or via a non-return valve, for mixing air with the oxygen for diluting the concentration of oxygen. Typically, the secondary supply of oxygen is delivered to the venturi chamber to compensate for the dilution of oxygen by the ambient air which is being drawn into the venturi chamber by the primary oxygen being supplied from the primary oxygen supply tube. A flow regulator valve may be provided in the primary oxygen supply to the venturi chamber for independently controlling the flow rate of the oxygen entering the venturi chamber. Also, a second flow regulator valve may be provided to regulate the secondary oxygen supply to the venturi chamber for controlling the concentration of oxygen in the oxygen/air mix.

U.S. Pat. No. 6,634,356 incorporates a pressure regulator means to a conventional gas mixing device as discussed above. According to U.S. Pat. No. 6,634,356, the pressure regulator means maintains the pressure of the oxygen being delivered to the primary and the secondary oxygen supply, and varies the flow rate of the oxygen/air mix delivered from the main outlet port independently of the ratio of the oxygen to air of the oxygen/air mix. Hence the oxygen/air ratio may be adjusted independently of the primary oxygen source's flow rate that allows the user to adjust flow and oxygen/air mix of the patient independently.

However, such a controller has disadvantages of its own. It is not possible to accurately determine the exact total flow of the oxygen/air mix exiting the venturi chamber because no flow meter is incorporated in the controller to determine the total flow of the oxygen/air mix. Instead, a separate flow meter may be incorporated at the end of the chamber where the oxygen/air mix exits. Flow meters generally restrict flow to some extent. Most flow meters that are inexpensive in construction restrict flow considerably. Venturi devices are very sensitive to flow restriction in their performance. It is, therefore, costly in construction of the device, or in the performance of the device, to incorporate a flow meter downstream of the venturi apparatus to approximate flow delivered to the patient.

There is therefore a need for a gas delivery device with a flow meter incorporated in the device such that performance is not compromised, and cost of construction is not excessive, which effectively allows the user to approximate the total flow of the oxygen/air mix being delivered.

### SUMMARY OF THE INVENTION

The present invention is an improved gas delivery apparatus for supplying a regulated oxygen/air mix to a patient.

According to the present invention, the gas delivery apparatus comprises a venturi chamber for blending oxygen and air to form the oxygen/air mix. The venturi chamber further includes a main oxygen nozzle portion disposed at one end of the chamber, a secondary oxygen inlet portion disposed laterally with the chamber. An ambient air inlet portion is disposed laterally with the chamber, and a gas delivery portion is disposed at the opposite end of the chamber for directing the oxygen/air mix to a patient. A pressurized oxygen source and a primary oxygen supply tube are in communication with the main oxygen nozzle portion for supplying oxygen from the pressurized oxygen source to the venturi chamber. A flow meter is disposed in the primary oxygen supply tube for measuring the flow of the pressurized oxygen through the main oxygen nozzle portion. As will be explained more fully in detail, one of the benefits in the present invention is that the flow meter, positioned in the primary oxygen supply tube, approximates the total flow of the oxygen/air mix delivered to the patient from the flow passing through the main oxygen nozzle. A first flow adjustment valve is disposed on the primary oxygen supply tube, either before or after the flow meter, for adjusting the flow of oxygen from the pressurized oxygen source to the main oxygen nozzle portion independently of the flow of oxygen from the pressurized oxygen source through the secondary oxygen inlet. A secondary oxygen supply tube is in communication with the secondary oxygen inlet portion for supplying additional oxygen from the pressurized oxygen source to the venturi chamber for enriching the oxygen concentration. A second flow adjustment valve is disposed on the secondary oxygen supply tube for adjusting the flow of oxygen from the pressurized oxygen source to the venturi chamber via the secondary oxygen inlet portion independently of the flow of oxygen from the pressurized oxygen source through the primary oxygen supply tube. An oxygen analyzer coupled with an oxygen sensor is in communication with the gas delivery portion for monitoring the oxygen concentration in the oxygen/air mix delivered to a patient. Lastly, a diffuser extending from the venturi chamber and expanding in cross-section from the venturi chamber causing the oxygen/air mixture to decrease in velocity and increase in driving pressure.

Accordingly, one of the objects of the present invention is to provide a flow meter on the primary oxygen supply line, upstream of the venturi chamber, to determine the total flow of the oxygen/air mix, without compromising the simplicity of such apparatus and the performance of the venturi effect.

As will be apparent in the present invention, one of the advantages is that a flow meter integrated upstream of the venturi allows for an accurate measurement of the oxygen flow using inexpensive, flow restrictive measuring means where gas driving pressure is abundant from the pressurized oxygen source. This flow is used to approximate the total flow of oxygen/air mix delivered through the gas delivery portion—eliminating a need for more expensive and less restrictive flow measuring means downstream of the venturi, thereby saving costs while maintaining performance.

It is contemplated that the present invention is generally appropriate for, but not limited to, oxygen therapy, obstructive sleep apnea therapies, CPAP therapy, neonatal and other ventilation systems and other respiratory therapies.

These and other benefits of the present invention will become more readily apparent after a review of the detailed description and preferred embodiments.

### BRIEF DESCRIPTION OF THE DRAWINGS

The invention will be further explained by reference to the accompanying drawings in which:

FIG. 1 is a schematic illustration of the principal features of an oxygen/air mixer according to the present invention.

### DETAILED DESCRIPTION OF THE INVENTION

While this invention is susceptible of embodiment in many different forms, there is shown in the drawings and will be described in detail below, specific embodiments with the understanding that the present disclosure is to be considered as an exemplification of the principles of the invention and is not intended to limit the invention to the embodiments illustrated.

This system, in accordance with the present invention, is a high flow delivery system to deliver mixed oxygen/air gas at flow ranging from zero up to in excess of 100 LPM. It is contemplated that the parameters of the present invention are appropriate for a gas delivery device such as oxygen therapy, obstructive sleep apnea therapies, CPAP therapy, neonatal and other ventilation systems and other respiratory therapies.

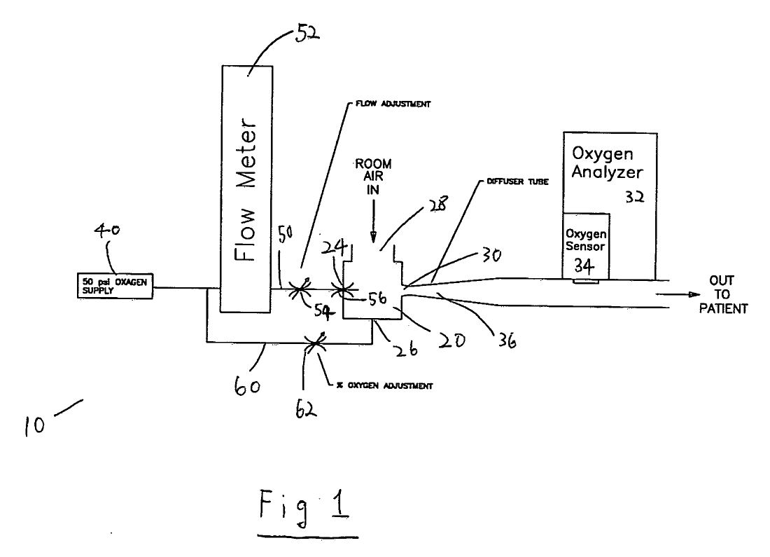

Now referring to FIG. 1 of the present invention, a gas flow apparatus 10 for supplying a regulated oxygen/air mix to a patient comprises a venturi chamber 20 for blending oxygen and air to form the oxygen/air mix. The venturi chamber 20 includes a main oxygen nozzle portion 24 disposed at one end of the chamber 20, a secondary oxygen inlet portion 26 disposed laterally with the chamber 20, an ambient air inlet portion 28 disposed laterally with the chamber 20, and a gas delivery portion 30 at the opposite end of the chamber 20 for directing the oxygen/air mix to a patient.

The ambient air inlet portion 28, as shown in FIG. 1, is sized such that ambient air is continuously in communication with the inlet portion 28 into the chamber 20. It is also contemplated that the ambient air inlet portion 28 may be suitably sized to fit various shapes of viral/bacterial filters (not shown).

The apparatus may be provided with various clamps, locking connections or friction connections to be used to connect the gas delivery portion 30 to the respiratory circuit (not shown).

A pressurized oxygen source 40 is connected to a primary oxygen supply tube 50, and the primary oxygen supply tube 50 is in communication with the main oxygen nozzle portion 24, wherein oxygen from the pressurized oxygen source 40 is delivered through the primary oxygen supply tube 50, through the main oxygen nozzle 24 to the venturi chamber 20.

As shown in FIG. 1, a flow meter 52 is positioned in the primary oxygen supply tube 50. The positioning of the flow meter, in the pressurized line, allows accurate measurement of the flow passing through the main oxygen nozzle 24, without the need for a low-resistance flow-metering technology. The total flow passing through the gas delivery portion 30 is approximated from the flow passing through the flow meter 52. Further, the indicating scale (not shown) printed on the flow meter 52 indicates the total flow of the oxygen/air mix rather than the flow of the pressurized oxygen. Table 1 below gives an example of the oxygen flow through the flow meter and the total flow that results, as well as the flow accuracy achieved.

In accordance with the present invention, the placement of the flow meter 52 in the upstream of the venturi device rather than in the downstream contemplates that any resistance caused by the flow meter 52 does not effect the performance of the venturi and, as a result, an accurate reading of the total flow of the oxygen/air mix can be measured without compromising venturi performance. Such configuration is beneficial in that an accurate measurement of the oxygen flow can be determined with a very inexpensive flow meter. Also, the flow meter 52 in the present invention can be utilized in wide variety of respiratory therapies, such as in oxygen therapy, obstructive sleep apnea therapies, CPAP therapy, neonatal and other ventilation systems.

Typically, the flow meter 52 is a Thorpe Tube type. An indicator ball floats in the Thorpe Tube and is elevated or floated in the Thorpe Tube by the flow of oxygen through a conical tapered tube. Operation of this type of flow meter is simplistic and is well known in the art, wherein, the level to which the ball is raised is indicative of the flow rate of gas through the meter. An indicating scale is converted to allow a user to read the flow rate of the total flow of the oxygen/air mix (see Table 1, below).

Table 1 shows the indicating scale on the flow meter 52 converting the flow rate of the oxygen into the flow rate of the oxygen/air mix.

| Flow rate | Oxygen flow rate | 5% accuracy | 10% accuracy |
|---|---|---|---|
| (LPM) | (LPM) | (LPM) | (LPM) |
| 10 | 1.38 | ±0.06 | ±0.12 |
| 20 | 2.44 | ±0.11 | ±0.23 |
| 30 | 3.63 | ±0.20 | ±0.38 |
| 40 | 5.02 | ±0.31 | ±0.61 |
| 50 | 6.59 | ±0.48 | ±0.93 |

The tolerance of ±5% and ±10% represents the accuracy in the flow meter scale printing between the flow reading and the actual flow delivered to a patient.

Also in FIG. 1, a first flow adjustment valve 54 is disposed on the primary oxygen supply tube 50 for adjusting the flow of oxygen from the pressurized oxygen source 40 to the main oxygen inlet portion 24 independently of the flow of oxygen from the pressurized oxygen source 40 through the secondary oxygen supply tube 50.

The first flow adjustment valve 54 may be upstream or downstream of the flow meter 52. However, positioning the first flow adjustment valve 54 downstream of the flow meter, as indicated in FIG. 1, is ideal as it allows the pressure inside the flow meter 52 to remain constant at the supply pressure, independent of the flow through the flow meter 52. Constant pressure inside the flow meter 52 results in improved accuracy of the flow meter 52.

As shown in FIG. 1, a secondary oxygen supply tube 60 is in communication with the secondary oxygen inlet portion 26 for supplying additional oxygen from the pressurized oxygen source 40 to the venturi chamber 20 for enriching the air to increase the oxygen concentration independently of the flow of oxygen from the pressurized oxygen source 40 through the secondary oxygen supply tube 60.

A second flow adjustment valve 62 is disposed on the secondary oxygen supply tube 60 for adjusting the flow of oxygen from the pressurized oxygen source 40 to the venturi chamber 20 via the secondary oxygen inlet portion 26 independently of the flow of oxygen from the pressurized oxygen source 40 through the secondary oxygen supply tube 60. This second flow adjustment valve 62 serves to control the oxygen concentration in the mix.

In accordance with the present invention, the arrangements of FIG. 1 may be modified, wherein, (i) the modification may consist in omission of the secondary oxygen supply tube and the secondary flow adjustment valve; (ii) the modification may consist of substitution of the primary flow adjustment valve with a fixed orifice to provide constant oxygen flows to the venturi chamber; (iii) the modification may consist of substitution of the secondary oxygen supply tube with a fixed orifice to continuously enrich the air in the venturi chamber; (iv) the modification may consist in omission of the primary flow adjustment valve; and (v) the modification may consist in omission of the primary flow adjustment valve, the secondary oxygen supply tube and the secondary flow adjustment valve.

Referring to FIG. 1, an oxygen analyzer 32 coupled with an oxygen sensor 34 is in communication with the gas delivery portion 30 for monitoring the oxygen concentration in the oxygen/air mix.

Also referring to FIG. 1, a diffuser 36 extends from the venturi chamber 20 and expands in cross-section from the venturi chamber 20 causing the oxygen/air mixture to decrease in velocity and increase in driving pressure.

The foregoing description merely explains and illustrates the invention and the invention is not limited thereto except insofar as the appended claims are so limited, as those skilled in the art who have the disclosure before them will be able to make modifications without departing from the scope of the invention.

1. An improved gas delivery apparatus for supplying a regulated oxygen/air mix to a patient, comprising:
a venturi chamber for blending oxygen and air to form the oxygen/air mix, including a main oxygen nozzle portion disposed at one end of the chamber, a secondary oxygen nozzle portion disposed laterally with the chamber, an ambient air inlet portion disposed laterally with the chamber, and a gas delivery portion at the opposite end of the chamber for directing the oxygen/air mix to a patient;
means for connecting to a pressurized oxygen source;
a primary oxygen supply tube in communication with the main oxygen nozzle portion for supplying oxygen from the pressurized oxygen source to the venturi chamber;
a flow meter disposed in the primary oxygen supply tube;
a first flow adjustment valve disposed on the primary oxygen supply tube for adjusting the flow of oxygen from the pressurized oxygen source to the main oxygen nozzle portion;
a secondary oxygen supply tube in communication with the secondary oxygen inlet portion for supplying additional oxygen from the pressurized oxygen source to the venturi chamber for enriching the air to increase the oxygen concentration; and,
a second flow adjustment valve disposed on the secondary oxygen supply tube for adjusting the flow of oxygen from the pressurized oxygen source to the venturi chamber via the secondary oxygen inlet portion independently of the flow of oxygen from the pressurized oxygen source through the primary oxygen supply tube.

2. The apparatus according to claim 1, wherein an indicating scale is provided in association with the flow meter for indicating the actual flow of the oxygen/air mix exiting the gas delivery portion and to a patient.

3. The apparatus according to claim 1, wherein the ambient air inlet portion is shaped to fit a filter of any size and shape on the air inlet portion.

4. The apparatus according to claim 1, wherein an oxygen analyzer and an oxygen sensor are integrally in communication with the gas delivery portion for monitoring the oxygen concentration in the oxygen/air mix delivered to a patient.

5. The apparatus according to claim 1, wherein a diffuser extends from the gas delivery portion of the venturi chamber and expands in cross-section from the venturi chamber for improving the driving pressure of the oxygen/air mixture produced by the venturi.

6. The apparatus according to claim 1, wherein the secondary oxygen supply tube and the secondary flow adjustment valve are not present.

7. The apparatus according to claim 1, wherein the secondary flow adjustment valve is replaced with a fixed orifice.

8. The apparatus according to claim 1, wherein the primary flow adjustment valve is not present.

9. The apparatus according to claim 1, wherein the primary flow adjustment valve is replaced with a fixed orifice.

10. The apparatus according to claim 1, wherein the primary flow adjustment valve, the secondary oxygen supply tube and the secondary flow adjustment valve are not present.
